(19) United States
(12) Patent Application Publication
(10) Pub. No.: US 2006/0127903 A1
(43) Pub. Date: Jun. 15, 2006

(54) Marker for cancer

(76) Inventors: Nuzhat Ahmed, Yallambie (AU); Gregory Rice, Warranwood (AU); Michael Quinn, Clifton Hill (AU)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/525,150

(22) PCT Filed: Aug. 20, 2003
(86) PCT No.: PCT/AU03/01058

§ 371 Date: Oct. 28, 2005

(30) Foreign Application Priority Data
Aug. 20, 2002 (AU) 2002951561

(51) Int. Cl. C12Q 1/68; G01N 33/574; C07H 21/04; C12P 21/06; C12N 9/12; C07K 16/40

(52) U.S. Cl. 435/6; 435/7.23; 435/69.1; 435/194; 435/320.1; 435/325; 530/388.26; 536/23.2

(57) ABSTRACT

The present invention relates to a cell-free immunoreactive Integrin Linked Kinase which is present in samples of biological fluids from patients suffering from ovarian cancer. The invention also relates to methods of diagnosis, screening, monitoring treatment and treatment of cancers, especially ovarian cancer, and to kits and compounds for use therein.

The present invention relates to a marker for cancer and use of this marker in methods of diagnosis and monitoring and treatment of cancer. The marker and methods particularly, although not exclusively, relate to ovarian cancer.

### BACKGROUND OF THE INVENTION

The goal of cancer screening is to detect pre-cancerous lesions or early stage malignancies in the asymptomatic pre-clinical phase of the disease so that subsequent diagnosis and treatment will have a significant impact on reducing cancer morbidity and mortality.

Epithelial ovarian cancer is a major cause of cancer morbidity and mortality in women. Serum biomarkers may provide a useful and cost-effective tool to detect asymptomatic ovarian cancer at an early stage. The identification of serum biomarkers suitable and specific for early stage detection and diagnosis holds great promise to improve the clinical outcome of patients. It is particularly important for patients presenting at an early stage with vague or no symptoms or with tumors that are relatively inaccessible to physical examination. The five-year survival for women diagnosed with early stage ovarian cancer is 90% while it is only 40% for women diagnosed at late stage (stage 3 or 4). Despite considerable effort directed at early stage screening no cost effective screening test has been developed and 75% of women still present with disseminated disease at diagnosis.

To date, no definite marker of ovarian cancer has been identified which is suitable for early-stage screening purposes. Cancer antigen 125 (CA 125) is the best-characterized serum marker for advanced stage ovarian cancer (Lavin, P. T. et al., (1987) Obstet Gynecol. 69:223-7). CA 125 is a 700 kDA glycoprotein expressed on the surface of cells. Its concentration in serum may be elevated in benign conditions and is elevated in >90% of patients with stage 3 and 4 ovarian cancer. CA 125 is not elevated in the serum beyond its cut off limit of 35 U/ml and accordingly only 50% of patients with stage 1 disease show elevated levels. Hence, CA 125 has insufficient sensitivity and specificity to be used as a population-based screening marker for early detection and diagnosis. Recent efforts in using CA 125 in combination with additional tumor markers in a longitudinal risk of cancer model (Skates, S. J. et al., (1995) Cancer 76:2004-10), and in combination with ultrasound as a second-line test have shown promising results in improving overall test specificity (Menon, U., (2000) BJOG. 107: 165-9) but do not provide a simple and reliable approach to diagnosis of ovarian cancer.

As a significant portion of ovarian cancer patients do not achieve complete remission and experience relapse, the evaluation of the effectiveness of therapy, using a reliable biomarker, is important. Moreover, as the early detection of relapse has a significant impact on the course of the disease and on the choice of second line of treatment it is crucial to identify a serum marker that depicts the clinical condition of a patient with significant reliability. Hence, there is a critical need for new serological tumor markers that individually or in combination with other markers may deliver the required sensitivity and specificity for both early detection and treatment monitoring.

The ability of tumor cells to invade locally and metastasize to distant organs is a major determinant of tumor progression. Invasion and subsequent dissemination require loss of adhesion between tumor cells and extracellular matrix. These processes are mediated by a large family of transmembrane receptors called integrins.

Integrins are intimately associated with the cytoskeleton through the cytoplasmic domain of the β subunit.

Integrin linked kinase (ILK) is a serine-threonine kinase and has been demonstrated to associate with the cytoplasmic domains of integrin β1 and β3 (Dedhar, S. et al., (1999) Trends Cell Biol. 9: 319-23). The nucleotide and protein sequence of ILK are known (Dedhar 1999 supra), it being a putative 59 kDa protein kinase with four ankyrin repeats at its N terminus. ILK is a ubiquitous protein and is expressed in a broad range of human tissues and cells of all lineages, including freshly-isolated monocytes and peripheral blood lymphocytes (Hannigan and Dedhar (1997 J. Mol. Med. 75(1): 35-44)). In cancer tissues, ILK expression is known to be stimulated by the protooncogene erbB2 in hyperplastic epidermis, and its expression is elevated in Ewing's sarcoma, primitive neuroectodermal tumor, medulloblastoma and neuroblastoma (Chung, D. H. et al., (1998) Virchows Arch. 433: 113-7). ILK expression in tissues has been shown to increase with the progression of prostate (Graff, J. R. et al., (2001) Clin. Cancer Res. 7: 1987-91) and colon tumor grade (Ito, R. et al., (2003) Virchows Arch. 442: 118-23).

To reduce the incidence of mortality from ovarian cancer, identification of markers which are detectable in blood or serum of cancer patients is essential to complement the use of existing tests in detecting early-stage disease.

### SUMMARY OF THE INVENTION

The inventors propose that ILK is a suitable marker of ovarian cancer in tissues and have carried out immunohistochemical studies to determine the relative expression of ILK in normal or benign tissues and in tissues from low-grade and high-grade (borderline; grade I/II and grade III) ovarian tumour of serous, mucinous, endometrioid and clear cell types.

As expected all carcinomas studied were positive for ILK and the staining intensity correlated significantly with the grade of tumour, indicating that like in prostate cancer, ILK expression is a marker for ovarian cancer and expression in ovarian tissue increases with the progression of the cancer.

When carrying out the above study the inventors surprisingly determined the presence of a new form of ILK.

Accordingly the present invention provides in a first aspect a cell-free immunoreactive form of Integrin Linked Kinase.

The immunoreactive Integrin Linked Kinase (irILK) according to the present invention may be characterised by a molecular weight of roughly 59 kDA as determined by SDS PAGE, being detectable in serum by Western blotting with a commercially available polyclonal antibody, (Upstate Biotechnology USA) raised against the kinase domain of ILK.

The irILK may also be detected (a) in peritoneal fluid by Western blotting with immunoaffinity-purified polyclonal anti-ILK corresponding to the kinase domain of human ILK (catalogue no. 06592, Upstate Biotechnology, USA) followed by peroxidase labelled secondary antibody, (b) in tissue-conditioned medium by Western blotting using ILK antibody; or (c) in serum or peritoneal fluid by immunoprecipitation on protein in 95% acetone:ethanol (1:1) with capture by antibody against ILK and immunoprecipitation visualised by Western blotting, the presence of irILK in any such sample being indicative of ovarian cancer.

The inventors detected irILK according to the first aspect of the invention in the serum of ovarian cancer patients. Previously ILK has only been detected in the tissues of cancer patients. Compared to serum from healthy volunteers and women with benign tumours the inventors have found that expression or irILK is significantly elevated in the serum of patients with ovarian cancer. The inventors have also detected irILK in the peritoneal fluid of cancer patients, and in tissue conditioned medium containing, or having contained ovarian tissue from patients with ovarian cancer.

The inventors propose the use of irILK as a biomarker for cancer. The inventors propose that irILK may be present in the serum of patients with cancers in which ILK has been implicated, that is Ewing's sarcoma, primitive neuroectodermal tumor, medulloblastoma and neuroblastoma (Chung, D. H. et al., (1998) Virchows Arch. 433: 113-7), prostate cancer (Graff, J. R. et al., (2001) Clin. Cancer Res. 7: 1987-91) and colon tumor (Ito, R. et al., (2003) Virchows Arch. 442: 118-23). The irILK may also be present in the serum of patients with cancers in which ILK is found to be implicated.

In a second aspect, the present invention provides a method of detection of cancer, comprising determining the presence or absence of irILK in a sample of a biological fluid from a subject, wherein the presence of irILK is an indication of cancer.

In a third aspect, the invention provides a method of monitoring the efficacy of a treatment for cancer, comprising carrying out periodic tests, each test comprising determining the concentration or activity of irILK in a sample of a biological fluid from a subject, wherein a decrease in irILK concentration or activity between tests is indicative of the efficacy of any treatment.

In a fourth aspect, the invention provides a method for detecting recurrence of cancer, comprising determining the presence or absence of irILK in a sample of a biological fluid from a subject who has had a cancer, wherein the presence of irILK indicates recurrence of the cancer.

In a fifth aspect, the invention provides a method of assessing the severity of a cancer, comprising quantitatively determining the amount or activity of irILK in a sample of a biological fluid from a subject, and correlating results with those previously determined for various grades or stages of the cancer or correlating results with one or more other markers of the cancer.

Preferably the other marker is CA125.

In a sixth aspect the present invention provides a kit for carrying out any one of the methods according to the methods of the second to fifth aspects of the invention, said kit comprising means for detecting irILK in biological fluid. Preferably the kit comprises an anti-ILK antibody.

Use of irILK according to the first aspect of the invention or a kit according to the sixth aspect of the invention in the diagnosis of cancer and study of treatment of a cancer is also provided, as is the use of irILK according to the first aspect of the invention or a kit according to the sixth aspect of the invention in the manufacture of a medicament for use in the diagnosis and study of treatment of a cancer.

In a seventh aspect the present invention provides a method of treatment of a cancer comprising administering to a patient determined as suffering therefrom an agent capable of blocking the increased expression of ILK, or the effect of increased expression of ILK.

In an eighth aspect the invention also provides a method for prevention of the onset of a cancer comprising administering to patients at risk of developing the cancer an agent capable of blocking increased expression of ILK, or the effect of increased expression of ILK.

In the seventh and eighth aspects of the invention the agent capable of blocking the increased expression of ILK may comprise an antisense nucleic acid for use in a gene therapy approach. The agent capable of blocking the effect of increased expression of ILK may be an anti-ILK antibody (preferably an antibody specific for ILK) or an antagonist of ILK activity.

Agents capable blocking the increased expression of ILK, or the effect of increased expression of ILK may be used in the manufacture of medicaments for use in treating a cancer.

The methods, uses and kits according to the second to eighth aspects of the invention are applicable to any cancer but are particularly applicable to cancers in which ILK has been implicated, including Ewing's sarcoma, primitive neuroectodermal tumor, medulloblastoma and neuroblastoma (Chung, D. H. et al., (1998) Virchows Arch. 433: 113-7), prostate cancer (Graff, J. R. et al., (2001) Clin. Cancer Res. 7: 1987-91) and colon tumor (Ito, R. et al., (2003) Virchows Arch. 442: 118-23). The irILK may also be present in the serum of patients with cancers in which ILK is found to be implicated.

The methods, uses and kits according to the second to eighth aspects of the invention are particularly applicable to ovarian cancer.

### DETAILED DESCRIPTION OF THE INVENTION

The inventors have found that irILK can be detected in biological fluids from patients with ovarian cancer, even in early stage ovarian cancer (as confirmed by Western Blotting). Thus irILK provides a candidate for development as a biomarker.

The inventors demonstrate that serum irILK expression was decreased by six cycles of chemotherapeutic treatment and was complementary to the change of CA 125 values before and after treatment. This finding makes irILK an ideal candidate for screening for cancer. The data suggest irILK may have potential clinical value as a candidate marker for screening and in monitoring response to therapy in cancer.

The irILK according to the first aspect of the invention is said to be “cell free”. This means that the protein is not merely contained within cells but is secreted from intact cells into medium perfusing the cells. This is in contrast to known intracellular ILK which may be detected in tissues or after cell lysis.

Whilst not wishing to be bound by theory the inventors propose that irILK is a secretory product of tumours. The inventors also propose that irILK may be present in blood cells, especially lymphocytes, neutrophils and monocytes.

The irILK according to the first aspect of the invention is said to be “immunoreactive”. This means that the irILK is capable of eliciting an immune response and is able to be detected by antibodies.

The irILK according to the present invention may also be termed soluble ILK, meaning that the ILK is present in solution in biological fluids rather than being confined within a cell.

The irILK according to the present invention is a low abundance protein which has been difficult to isolate in sufficient quantities to sequence. However, the inventors have clearly shown methods for detecting irILK and that the presence of irILK in samples of biological fluid is linked to and indicative of ovarian cancer.

The expression of irILK may be detected by any suitable means. According to a preferred embodiment of the invention the method comprises an in vitro immunoassay arranged to detect irILK protein or specific fragments thereof. The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, chemiluminescence, precipitin reactions, gel diffusion precipitin reactions, immunodiffusion assays, agglutination assays, complement-fixation assays, immunoradiometric assays, fluorescent immunoassays, protein A immunoassays, to name but a few.

Preferably a specific binding partner for irILK or fragments thereof can be used quantitatively to show levels of irILK in a sample. The specific binding partner is preferably functional, producing a label, or has a label attached thereto to show the presence of and amount of irILK. Examples of specific binding partners for irILK include the ILK substrates (and fragments) and anti-ILK antibodies, although other suitable specific binding partners will be apparent to those skilled in the art.

Preferably antibodies raised against the whole irILK protein or specific fragments thereof are used in immuno assays as specific binding partners for irILK. Specific antibodies directed against intracellular ILK are commercially available, and may be used in such methods include protein A purified polyclonal anti-ILK against the kinase domain, cat no 06-550, protein G-purified monoclonal against full-length GST-ILK fusion protein, cat no 05575, and immunoaffinity purified polyclonal against the kinase domain of ILK, cat no 06592 (all available from Upstate Biotechnology, USA). Another suitable antibody is available from StressGen Biotechnologies Corp, Canada is a polyclonal against Gst-fusion full length ILK, cat no KAP-ST203.

Other ways of determining the presence of irILK protein in a sample are by molecular weight, densitometry or charge. Chromatography on a porous carrier or SDS PAGE may be used to show levels of irILK in a sample due to the distance travelled along the carrier. Isoelectric focussing may be used to identify irILK due to its charge.

According to an alternative embodiment of the invention the method comprises an in vitro assay arranged to detect irILK nucleic acid or fragments thereof. Preferably this in vitro assay comprises hybridisation, sequencing or amplification techniques such as PCR and real time PCR.

ILK genes and related nucleic acid sequences and subsequences, including complementary sequences, and other gene sequences, can also be used in hybridization assays.

ILK nucleic acid sequences, or subsequences thereof comprising about at least 8 nucleotides, can be used as hybridization probes. Hybridization assays can be used to detect, prognose, diagnose, or monitor cancers associated with increased ILK expression. In particular, such a hybridization assay is carried out by a method comprising contacting a sample containing nucleic acid with a nucleic acid probe capable of hybridizing to ILK DNA or RNA, under conditions such that hybridization can occur, and detecting or measuring any resulting hybridization.

As when assaying for either the irILK protein or nucleic acid in some of the aspects of the invention, it is preferable to show the concentration or activity of irILK and accordingly it is preferable that the in vitro assay is quantitative. The reagents and vessels necessary for assaying irILK levels may be provided in a kit.

According to the second and fourth aspects of the invention any irILK detected (even a small, seemingly negligible amount) is taken to show the “presence” of irILK and accordingly provides a diagnosis of cancer (or of the recurrence of a cancer). In the same way if no irILK is detected this is taken to show the “absence” of irILK and accordingly provides a diagnosis of no cancer (or no recurrence of the cancer).

The third aspect of the invention provides a way of monitoring the efficacy of a treatment regime for cancer. “Efficacy” defines whether or not the treatment is having an effect on the cancer, whether the cancer is being treated, getting worse or remaining the same. The first time the test is carried out may be before treatment is started, with subsequent tests determining if the treatment has any effect.

The fifth aspect of the invention addresses assessing the severity of cancer. Amounts of irILK in biological samples have been shown by the inventors to correlate well with the progression of ovarian cancer as determined by other means, for example by determining the amount of ILK (intracellular) in tissues by histology or other means. Additionally the amount of irILK detected correlates well with the amount of CA 125 (in samples in which CA 125 testing is useful). Accordingly it is proposed that by quantifying the amount of irILK detected and comparing this with known data for ILK or CA 125 already determined, that the stage or grade at which the cancer has reached may be determined. This aspect is more clearly explained with reference to the examples.

It is preferred that the methods of the present invention is performed yearly, more preferably, every six months, even more preferably quarterly and most preferably every six weeks. The number of tests taken per year would vary depending on the degree of risk associated with the subject and the results of any previous test.

The methods according to the present invention may be carried out on whole blood, plasma, serum or peritoneal fluid, ascites, or on medium used to perfuse ovarian biopsies (tissue conditioned medium). A person skilled in the art will readily be able to determine whether other biological fluids, such as urine, tears, sputum, saliva or synovial fluid samples could also be used; however, it will be appreciated that in these cases more sensitive detection methods may be required.

Optionally the methods of the invention also comprise the step of determining levels of another cancer marker, such as CA125, in the biological fluid, using conventional methods.

The methods of treatment or prophylaxis of cancer according to the seventh and eighth aspects of the invention involves administering to a patient an agent capable of blocking an increased expression of ILK, or the effect of an increased expression of ILK.

The agent capable of blocking the increased expression of ILK may comprise an antisense nucleic acid for use in a gene therapy approach. The present invention provides the therapeutic or prophylactic use of nucleic acids of at least six nucleotides that are anti sense to a gene or cDNA encoding ILK or a portion thereof. “Antisense” as used herein refers to a nucleic acid capable of hybridizing to a portion of ILK RNA (preferably mRNA) by virtue of some sequence complementarity. Such antisense nucleic acids have utility as agents to block ILK expression, and can be used in the treatment or prevention of cancer.

The antisense nucleic acids of the invention can be oligonucleotides that are double-stranded or single-stranded, RNA or DNA or a modification or derivative thereof, which can be directly administered to a cell, or which can be produced intracellularly by transcription of exogenous, introduced sequences.

In a specific embodiment, the ILK antisense nucleic acids provided by the invention are used for the treatment of cancers in which ILK expression is elevated.

Double stranded RNA interference has been used to knock out ILK expression (Troussard AA et al., (2003) J. Biol. Chem. 278: 22374-8) and this technique could be used to treat cancer. Another way would be to introduce a kinase dead mutant by a gene delivery technique such as a retroviral system, for example Glu in the 359 position of the kinase domain may be replaced by lysine making serine/throenine kinase activity of ILK inactive (Hannigan GE et al, (1996) Nature 379, 91-96).

The antisense nucleic acids of the invention comprise a sequence complementary to at least a portion of an RNA transcript of an ILK gene, preferably a human ILK gene. However, absolute complementarity, although preferred, is not required. A sequence “complementary to at least a portion of an RNA,” as referred to herein, means a sequence having sufficient complementarity to be able to hybridize with the RNA, forming a stable duplex; in the case of double-stranded ILK antisense nucleic acids, a single strand of the duplex DNA may thus be tested, or triplex formation may be assayed. The ability to hybridize will depend on both the degree of complementarity and the length of the anti sense nucleic acid. Generally, the longer the hybridizing nucleic acid, the more base mismatches with ILK RNA it may contain and still form a stable duplex (or triplex, as the case may be). One skilled in the art can ascertain a tolerable degree of mismatch by use of standard procedures to determine the melting point of the hybridized complex.

The agent capable of blocking the effect of increased expression of ILK may be an anti-ILK antibody (preferably an antibody specific for ILK) or an antagonist of ILK activity. Specific inhibitors of ILK activity are currently in clinical trials by Kinetek Pharmaceuticals, Inc. (Yoganathan A et al., (2002) Pharmacology & Therapeutics 93: 233-242).

Various delivery systems are known and can be used to administer an agent blocking the effect of increased expression of ILK, e.g., encapsulation in liposomes, microparticles, microcapsules, expression by recombinant cells, receptor-mediated endocytosis, construction of a agent nucleic acid as part of a retroviral or other vector, and so on. Methods of introduction include but are not limited to intradermal, intramuscular, intraperitoneal, intravenous, subcutaneous, intranasal, and oral routes. The agents may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In a specific embodiment, it may be desirable to administer the agents locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, nonporous, or gelatinous material, including membranes, such as sialastic membranes, or fibers. In one embodiment, administration can be by direct injection at the site (or former site) of a malignant tumor or neoplastic or preneoplastic tissue.

Where the agent is a nucleic acid, the nucleic acid can be administered in vivo to promote expression of its encoded protein, by constructing it as part of an appropriate nucleic acid expression vector and administering it so that it becomes intracellular, e.g., by use of a retroviral vector, or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus. Alternatively, a nucleic acid agent can be introduced intracellularly and incorporated within host cell DNA for expression, by homologous recombination.

The amount of the agent suitable in the treatment of a particular cancer will depend on the nature of the nature, and can be determined by standard clinical techniques. In addition, in vitro assays may optionally be employed to help identify optimal dosage ranges. The precise dose to be employed in the formulation will also depend on the route of administration, and the seriousness of the cancer, and should be decided according to the judgment of the practitioner and each patient's circumstances. However, suitable dosage ranges for intravenous administration are generally about 20-500 micrograms of active agent per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

Suppositories generally contain active ingredient in the range of 0.5% to 10% by weight; oral formulations preferably contain 10% to 95% active ingredient.

The inventors have show that the cancer that may be diagnosed, monitored or treated according to preferred embodiments of the invention is ovarian cancer.

A neoplasm or tumour is a neoplastic mass resulting from abnormal uncontrolled cell growth, which may cause swelling on the body surface, and which can be benign or malignant. Benign tumors generally remain localized. Malignant tumors are collectively termed cancers. The term “malignant” generally means that the tumor can invade and destroy neighboring body structures and spread to distant sites to cause death (for review, see Robbins and Angell, 1976, Basic Pathology, 2d Ed., W.B. Saunders Co., Philadelphia, pp. 68-122).

The invention may be applicable to all cancers in which irILK is present and may be used to diagnose cancer, cancer progression, recurrence and the efficacy of cancer treatment and inhibitors of ILK may be used to treat a cancerous condition or to prevent progression from a pre-neoplastic or non-malignant state (e.g. metaplastic condition) into a neoplastic or malignant state.

The inventors propose that irILK may be present in the serum of patients suffering from other cancers in which intracellular ILK expression is known, or will be found, to be increased, for example Ewing's sarcoma, primitive neuroectodermal tumor, medulloblastoma and neuroblastoma (Chung, D. H. et al., (1998) Virchows Arch. 433: 113-7), prostate cancer (Graff, J. R. et al., (2001) Clin. Cancer Res. 7: 1987-91) and colon tumor (Ito, R. et al., (2003) Virchows Arch. 442: 118-23).

Cancers that may involve increased expression of ILK include but are not limited to those listed below (for a review of such disorders, see Fishman et al., 1985, Medicine, 2d Ed., J. B. Lippincott Co., Philadelphia):

Malignancies and Related Disorders

Leukemia

acute leukemia

acute lymphocytic leukemia

acute myelocytic leukemia

myeloblastic

promyelocytic

myelomonocytic

monocytic

erythroleukemia

chronic leukemia

chronic myelocytic (granulocytic) leukemia

chronic lymphocytic leukemia

Polycythemia vera

Lymphoma

Hodgkin's disease

non-Hodgkin's disease

Multiple myeloma Waldenström's macroglobulinemia

Heavy chain disease

Solid tumors

sarcomas and carcinomas

fibrosarcoma

myxosarcoma

liposarcoma

chondrosarcoma

osteogenic sarcoma

chordoma

angiosarcoma

endotheliosarcoma

lymphangiosarcoma

lymphangioendotheliosarcoma

synovioma

mesothelioma

Ewing's tumor

leiomyosarcoma

rhabdomyosarcoma

colon carcinoma

pancreatic cancer

breast cancer

ovarian cancer

prostate cancer

squamous cell carcinoma

basal cell carcinoma

adenocarcinoma

sweat gland carcinoma

sebaceous gland carcinoma

papillary carcinoma

papillary adenocarcinomas

cystadenocarcinoma

medullary carcinoma

bronchogenic carcinoma

renal cell carcinoma

hepatoma

bile duct carcinoma

choriocarcinoma

seminoma

embryonal carcinoma

Wilms' tumor

cervical cancer

testicular tumor

lung carcinoma

small cell lung carcinoma

bladder carcinoma

epithelial carcinoma

glioma

astrocytoma

medulloblastoma

craniopharyngioma

ependymoma

pinealoma

hemangioblastoma

acoustic neuroma

oligodendroglioma

menangioma

melanoma

neuroblastoma

retinoblastoma

The subject or source of cancer may be any mammal but is preferably a human, or may be a domestic or companion animal. While it is particularly contemplated that the compounds of the invention are suitable for use in medical treatment of humans, they are also applicable to veterinary treatment, including treatment of companion animals such as dogs and cats, and domestic animals such as horses, cattle and sheep, or zoo animals such as non-human primates, felids, canids, bovids, and ungulates.

For the purposes of this specification it will be clearly understood that the word “comprising” means “including but not limited to”, and that the word “comprises” has a corresponding meaning.

A preferred embodiment of the present invention will now be described in detail by way of reference only to the following non-limiting examples and drawings.

### BRIEF DESCRIPTION OF THE FIGURES

FIG. 1 shows the results of immunohistochemistry using a polyclonal antibody against ILK. No immunoreactive ILK was present in normal ovaries (A), but the expression of ILK in two grade III ovarian tumour tissues (serous, panel B and endometrioid, panel C) was detected.

FIG. 2 shows the expression of irILK in human serum. (a and b) show Western blotting of serum samples. (c) shows quantification of irILK expression by densitometry expressed as mean peak OD±SEM the number of samples for each group. (d) shows quantitative determination of CA 125 levels in matching serum samples (a and b), represented as a mean±SEM of the number of samples per group.

FIG. 3 shows the expression of irILK in ovarian tumours. (a) show Western blotting of tissue homogenates. (b) shows quantification of irILK expression by densitometry expressed as mean peak OD±SEM the number of samples for each group.

FIG. 4 shows the expression of irILK in PTF of ovarian cancer patients. (a) shows western blotting of PTF samples. b) shows quantification of irILK expression in PTF by densitometry expressed as mean peak OD±SEM the number of samples for each group.

FIG. 5 shows irILK expression in tissue conditioned medium by Western blotting and (b) quantification using densitometry.

FIG. 6 shows immunoprecipation of irILK in PTF and serum of patients with grade 3 ovarian cancer, visualised by Western blotting.

FIG. 7 shows the effect of chemotherapy on the expression of irILK in the serum of ovarian cancer patients. (a) Western blotting of serum samples from patients with grade 1 ovarian cancer before and after two (patient 3) and six cycles of carboplatin/taxol. (b) Quantification of irILK expression in the serum samples by densitometry and expressed as peak OD±SEM of the number of samples described above. (c) Quantitative determination of CA 125 levels in the matching serum samples of patients with grade 1 ovarian cancer.

### EXAMPLES

### Example 1

### Immunohistochemical Analysis of Ovarian Tissue

The immunohistochemical study utilised resected tissues not required for clinical analysis that were obtained from patients who presented for surgery at the Royal Women's Hospital, Melbourne, Australia, only with informed consent.

The pathology diagnosis and tumour grade were determined by two staff pathologists. The classification of the tumours was performed as part of the clinical diagnosis. Histological grading of ovarian cancer tumours was performed by the method described by Silverberg (Silverberg, S. G. (2000) Int. J. Gynecol. Pathol. 19: 7-15). Peritoneal fluid (PTF) was obtained from patients undergoing surgery after diagnosis of ovarian cancer.

The tissues were frozen in embedding medium (OCT) by immersion in isopentane cooled in dry ice. They were then stored at −80° C. until needed. Frozen sections of the tissues were cut at 5 μm thickness and if not used immediately, they were stored at −20° C. For staining, sections were fixed in cold acetone for 15 min and held in Tris buffer (100 mM, pH 7.6). Endogenous peroxidase activity was removed using 3% hydrogen peroxide in methanol and endogenous biotin activity was blocked using a sequence of diluted egg white (5% in distilled water) and skimmed milk powder (5% in distilled water), all for 10 min each. The sections were incubated for 1 hour in ILK polyclonal antibody (StressGen, Canada) diluted 1/200 in 1% BSA in Tris buffer (100 Mm, pH 7.6). Antibody binding was amplified using biotin and streptavidin HRP (DAKO, Denmark) for 15 min each and the complex was visualised using diaminobenidine (DAB). Nuclei were lightly stained with Mayer's heamatoxylin. An isotype IgG1, suitably diluted, was substituted for the antibody as a negative control.

Sections were assayed microscopically for positive DAB staining. The intensity of ILK expression was scored in a blind fashion as −,+,++ and +++ corresponding to negative, weak, moderate and strong immunoreactivity (Table 1). Four sections were assayed per tissue. The tissue and cellular distribution of staining was determined in addition to the type of staining. The percentage of tissues having weak, moderate and strong staining were scored for each particular grade and type. Parallel frozen sections were stained with haematoxylin and eosin to confirm the pathology diagnosis. Haematoxylin and eosin and immunostained, sections were reviewed independently by two observers to verify cell type, grade and immunohistochemical score.

The association between the intensity of the immunohistochemistry staining and the histological grade and type of tumour was determined by X² analysis using the SPSS statistical package (Coakes & Steed, (1996) SPSS for Windows—Analysis without Anguish. John Wiley: 153-7).

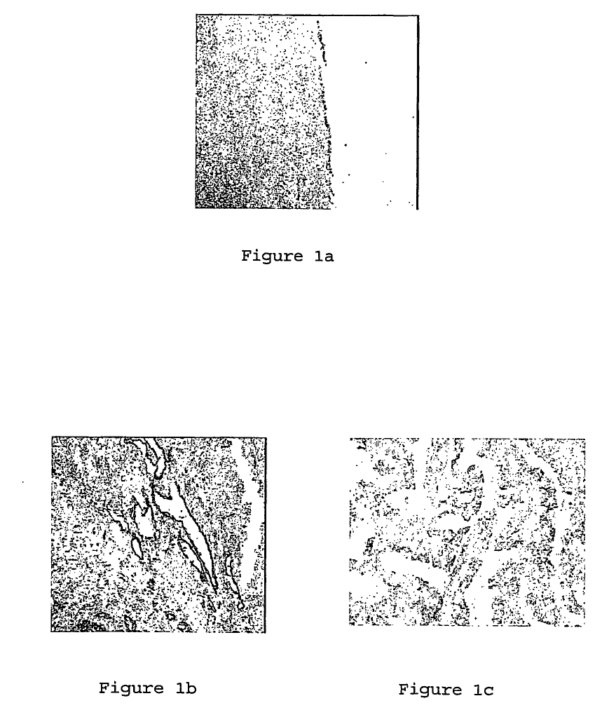

FIG. 1 shows an immunohistochemical comparison between samples of normal ovarian epithelium and of grade III serous and endometrioid ovarian tumours. No expression of ILK was observed in normal ovarian epithelium, as shown in FIG. 1A. In grade III serous and endometrioid ovarian tumours, shown in FIGS. 1B and 1C, high expression of ILK was detected in blood vessels lining the epithelium, but scattered epithelium staining was also observed.

The lack of expression of ILK in normal epithelium and expression of ILK in advanced stage tumours indicates that ILK is critical for ovarian cancer progression. This was not unexpected, given that ILK expression is elevated in many other cancers, including prostate cancer. The strong expression of ILK in endothelial cells and in blood vessels of grade III tumour indicates that ILK may be involved with angiogenesis.

TABLE 1
Intensity of ILK immunoreactivitY in 73 normal
ovarian and tumour tissues

| Histology | ILK Expression | No. of Patients |
|---|---|---|
| Normal | − | 10 |
| Benign serous | −/+ | 6 (3/3) |
| Borderline serous | ++/+++ | 6 (5/1) |
| Grade I/II serous | ++/+++ | 5 (2/3) |
| Grade III serous | ++/+++ | 14 (5/9) |
| Benign mucinous | ++/+++ | 4 (2/2) |
| Borderline mucinous | ++/+++ | 8 (6/2) |
| Grade I mucinous | ++/+++ | 4 (1/3) |
| Grade I/II endometroid | +/++ | 8 (4/4) |
| Grade III endometroid | ++/+++ | 5 (1/4) |
| Grade III clear cell | +++ | 2 |
| carcinoma | | |
| Grade III transitional | +++ | 1 |
| carcinoma | | |

An interesting observation as shown in Table 1 was the increment in ILK expression in ovarian tumour tissues as the cancer progresses, allowing ILK to be used as a marker for the progression of the cancer.

Another interesting observation was the detection of a cell-free, soluble form of ILK, termed immunoreactive ILK (irILK).

### Example 2

### Expression of a New Form of ILK in Biological Fluids from Ovarian Cancer Patients

Thirty six specimens, including normal (n=6), benign (n=6), borderline (n=4), grade 1 (n=5), grade 2 (n=5) and grade 3 (n=10) were evaluated for the expression of irILK by Western blotting. The expression of irILK was evaluated in peritoneal fluid (PTF, n=10) and benign cystic fluid (BCF, n=4). In addition, tissue-conditioned medium obtained from the cultures of primary ovarian tumors (n=9) was examined for the presence of irILK. Cell-free irILK was immunoprecipitated from the serum and PTF of patients with ovarian cancer.

The mean age of healthy volunteers participating in the study was 47 while that of women presenting with ovarian cancer was 62. Whole blood (10 ml) was collected by venepuncture into plain collection tubes, and allowed to clot at room temperature for 30 min. Samples were then centrifuged at 2000 g for 10 min after which serum was collected. An aliquot (100 μl) was removed for the determination of total protein. Serum was stored at −80° C. until analysed.

Resected tissues not required for clinical analysis were obtained from patients who presented for surgery at the Royal Women's Hospital, Melbourne, after the provision of a participant information statement and only with informed consent. Normal ovaries, needed for control comparisons were removed from patients undergoing surgery as a result of suspicious ultrasound images, palpable abdominal masses and family history. The pathology diagnosis and tumor grade was determined by two staff pathologists in the Department of Pathology, Royal Women's Hospital, Melbourne. The histological classification of the tumors was performed by the method described by Silverberg supra.

Protein Assay

Total protein content was determined using a commercial protein assay kit with BSA standards according to the manufacturer's instruction (Pierce, Rockford, Ill., USA).

Western Blotting

Serum samples containing equal amounts of protein (50 μg) were electrophoresed on 10% SDS-PAGE gels under non-reducing conditions and transferred to nitrocellulose membranes. Membranes were probed with immunoaffinity-purified polyclonal anti-ILK corresponding to the kinase domain of human ILK (Upstate Biotechnology, USA) followed by peroxidase-labelled secondary antibody and visualised by the ECL (Amersham, UK) detection system according to the manufacturer's instructions. The number of samples in each group was normal (n=6), benign (n=6), borderline (n=4), grade 1 (n=5), grade 2 (n=5) and grade 3 (n=10). The results are representative of one experiment repeated three times.

Quantification of irILK expression was performed by densitometry and expressed as mean peak OD±SEM of the number of samples described in each group. The experiment was repeated three times.

The quantitative determination of CA 125 levels in matching serum samples (a and b) was determined by using ACS:180 OV Automated Chemiluminescence Systems.

Preparation of Tissue-Conditioned Medium

Fresh ovarian tissue specimen were cut into small pieces (0.1 mg wet weight) and incubated in 3 ml of serum free RPMI-1640 supplemented with 2 mM glutamine (JRH Biosciences, Australia) and 100 μg/ml of penicillin/streptomycin (JRH Biosciences, Australia) for 48 h in humidified 5% CO₂ incubator. The tissue-conditioned medium was collected by centrifugation and concentrated 20-30-fold by using Biomax Ultrafree Centrifugal Filter Unit (Millipore, Bedford, USA) with a 10-kDa pore diameter cut-off. Samples with equal protein load were analysed by Western blotting to determine the expression of irILK in the tissue-conditioned medium of primary cultures.

Immunoprecipitation of irILK from Serum and Ascites of Cancer Patients

irILK from serum and ascites of ovarian cancer patients was immunoprecipitated by first isolating the protein from serum or ascites in 9 parts of 95% ethanol acetone (1:1) to 1 part of serum or ascites. After incubation overnight at −20° C., the proteins were precipitated by centrifugation at 400 g at 4° C. The precipitate was washed 3 times in 95% ethanol acetone (1:1) and dried in a speed vac centrifuge for 75 min. The dried protein was re-suspended in Tris-buffer (100 mM Tris-HCl, pH 7.5, 150 mM NaCl, 1 mM CaCl₂, 1% Triton X-100, 0.1% SDS, 0.1% NP-40, 1 mM vanadate, 1 μg/ml pepstatin, 1 mM PMSF, 5 μg/ml Trasylol and 1 μg/ml of leupeptin) and irILK was immunoprecipitated with antibody against ILK (Upstate Bitechnology, USA), or isotype matched control. The immune-complexes were isolated on protein A-Sepharose and released by the addition of 100 μl of Laemmli sample buffer heated for 5 min at 95° C. Isolated proteins were analyzed in 10% SDS-PAGE gel under non-reducing conditions and were transferred to nitrocellulose membranes. Membranes were probed with anti-ILk antibody followed by peroxidase-labelled secondary antibody. ECL was used to detect the protein bands.

Determination of CA 125 Value

CA 125 values were determined by using ACS:180 OV Automated Chemiluminescence Systems (Bayer, Germany). The ACS:180 OV assay is a two-site sandwich immunoassay using direct chemiluminescence technology that uses purified monoclonal antibodies specific for CA 125. A direct relationship exits between the amount of CA 125 in the patient sample and the amount of relative light units (RLUs) detected by the system.

Chemotherapeutic Treatment

Patients diagnosed with ovarian cancer were treated with combination therapy consisting of carboplatin (AUC 5)/taxol (175 mg/m²body weight) following surgery elevated. The combination drugs were given to patients every three weeks by intra-venous infusion. Each patient underwent six cycles of chemotherapy and CA 125 values were determined before and after each cycle.

Statistical Methods

Student's t-test was used for statistical analyses. Statistical significance was indicated by p<0.05. Data are presented as means±S.E.M. To determine variation of irILK expression in the serum of normal, benign and four groups of ovarian cancer patients (borderline, grade 1, grade 2 and grade 3) Friedman non-parametric two-way analysis and Kruskal Wallis one-way analysis was performed.

Results

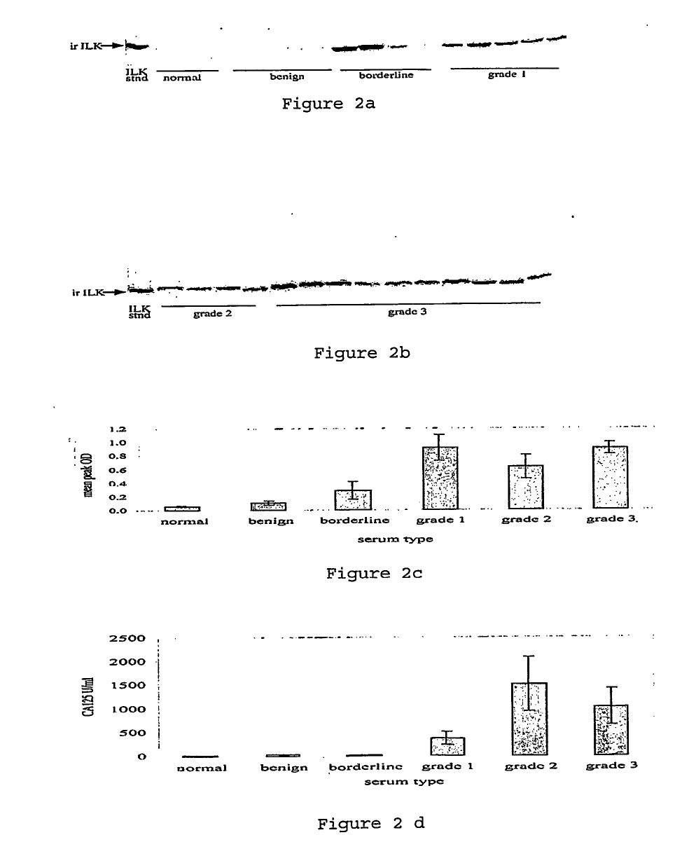

FIG. 2 shows the results of Western blotting. FIG. 2 (a and b) shows the expression of irILK in human serum. FIG. 2(c) shows the quantification of irILK expression performed by densitometry. Results are representative of one experiment. FIG. 2(d) shows the results of quantitative determination of CA 125 levels in matching serum samples (a and b). Results are represented as mean±SEM of the number of samples described in each group.

All four patients with borderline tumours had Stage I disease. Of the five patients with grade 1 cancer three had Stage I, one Stage II and one Stage III disease. All patients with grade 2 and grade 3 cancers had Stage III disease. The mean serum irILK expression for grade 1, 2 and 3 disease was six to nine-fold higher than in control subjects (p<0.01) (FIGS. 2 a, b and c). Notably women with borderline tumours had two-fold higher irILK expression than the control group. To determine variation of irILK expression in the serum of normal, benign and four groups of ovarian cancer patients (borderline, grade 1, grade 2 and grade 3) Friedman non-parametric two-way analysis and Kruskal Wallis one-way analysis was undertaken. The results indicate that there is a significant difference in the irILK expression among the groups (X²=14, df=5, p=0.016). The average association between variables was determined by Kendall's co-efficient of concordance=0.700. Similar results were obtained with Kruskal Wallis test (X²=16.06, df=5, p=0.007).

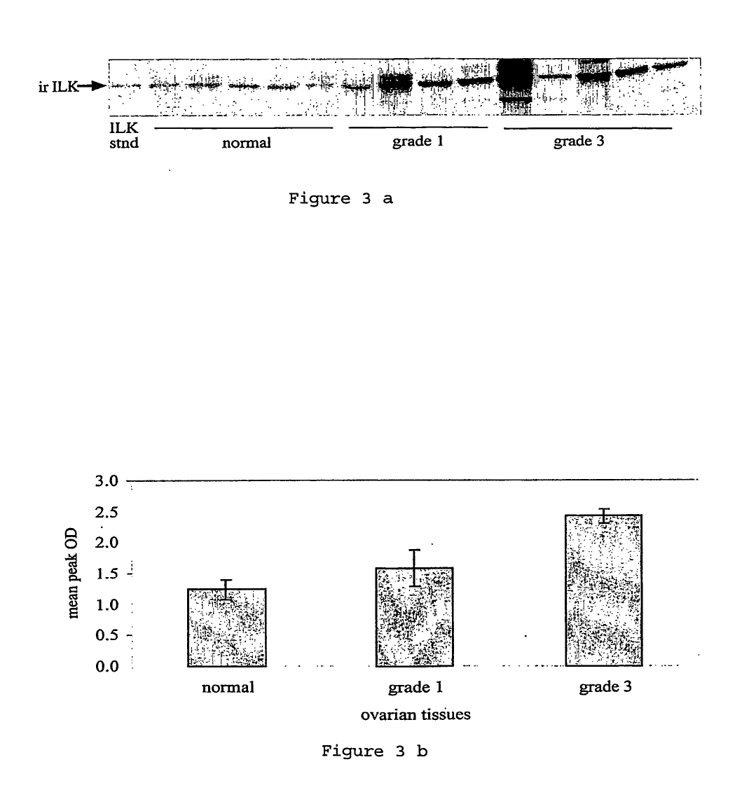

Enhanced serum expression of irILK in the patients with ovarian cancer correlated with elevated serum CA 125 concentrations (FIG. 2 d). This suggests that irILK behaves in a complementary fashion to CA 125 antigen with the progression of ovarian cancer. Enhanced expression of irILK in the serum of patients with ovarian cancer correlated with enhanced expression of ILK in the ovarian tumour tissues but it was not so pronounced as in the serum (FIGS. 3 a and b). Compared to normal ovaries, the expression of ILK was enhanced by 1.3-fold in grade 1 and 1.6-fold (p>0.01) in grade 3 ovarian tumour tissues (FIGS. 3 a and b). This is consistent with the up regulation of ILK we have recently demonstrated immunohistochemically in the different sub-types of ovarian tumours (n=73) (Ahmed et al, in press).

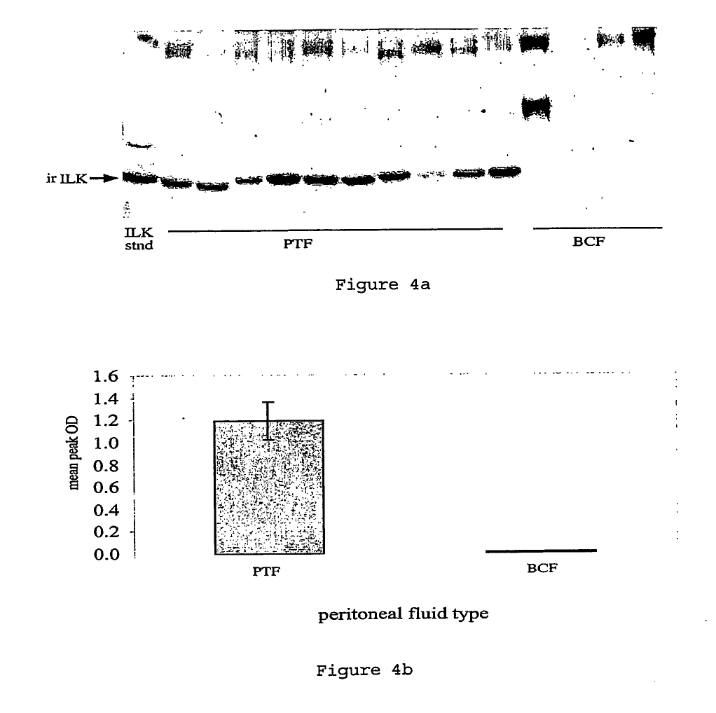

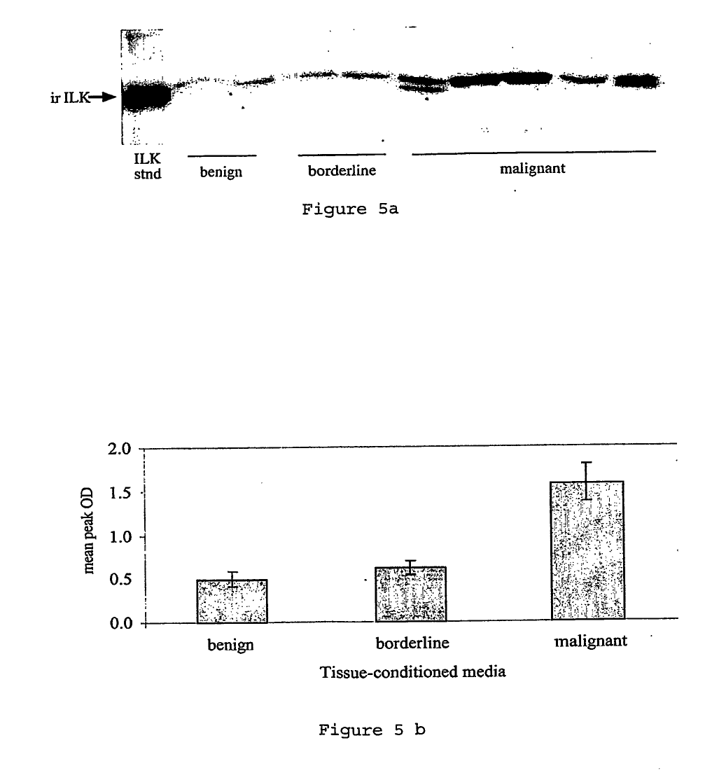

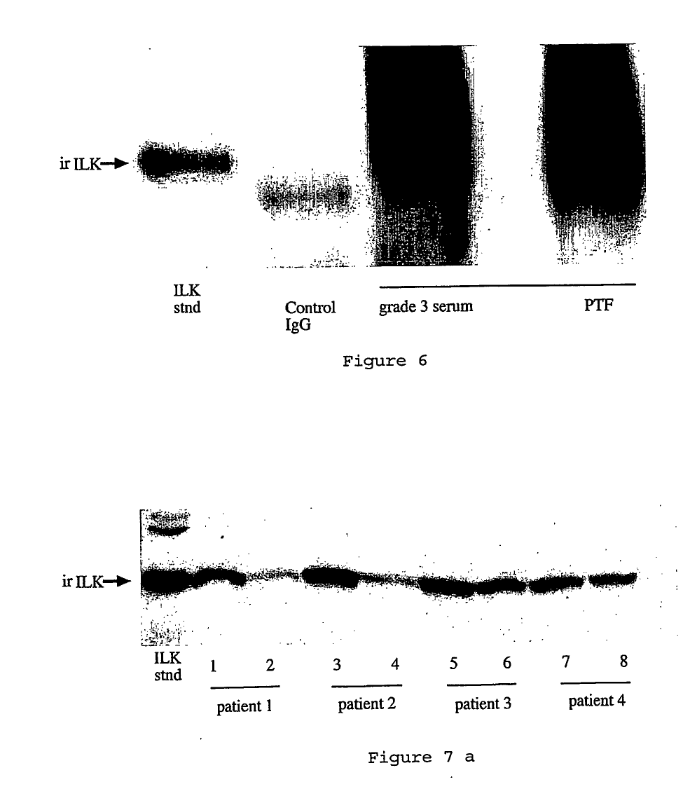

As peritoneum constitutes the tumour host microenvironment and is the first site to accumulate proteins produced by the tumour, we determined the expression of irILK in the peritoneal fluid of the cancer patients. Significant expression of irILK was present in the PTF of patients with grade 3 ovarian cancer (n=10) while no such expression can be detected in the BCF (n=4) obtained from patients presented with benign cysts of the ovaries (FIGS. 4 a and b, p<0.01). To determine if ovarian tumours contribute to the presence of irILK in the peritoneum and subsequently in the serum of cancer patients, the expression of irILK was determined in the tissue-conditioned medium (concentrated 20-25-fold) prepared from primary ovarian tumours obtained from benign, borderline and grade 3 cancer patients. Conditioned medium from benign and borderline tumours showed expression of irILK but it was eight-fold lower than the expression determined in the tissue-conditioned medium of malignant tumours (FIGS. 5 a and b, p<0.01). irILK was also immunoprecipitated from the serum and PTF of patients with grade 3 cancer (FIG. 6).

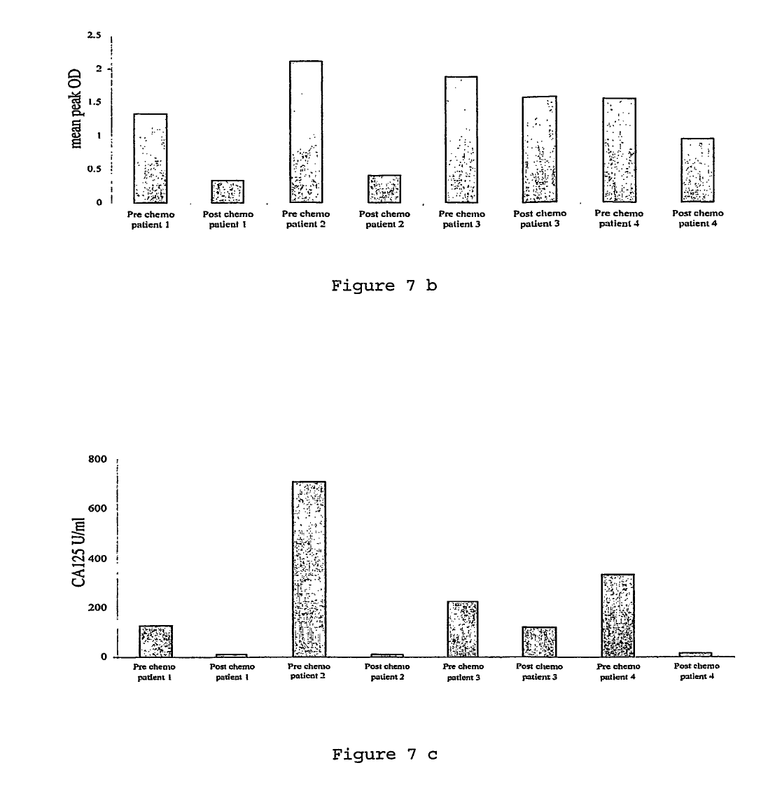

To determine if chemotherapeutic treatment had any affect on the expression of irILK in the serum of ovarian cancer patients, matching samples from patients were tested for ILK expression before and after six cycles of chemotherapy. The expression of irILK was reduced after six cycles of chemotherapeutic treatment and paralleled serum concentrations of CA 125 before and after treatment (FIGS. 7 a and b). Suppression of irILK expression after two cycles of chemotherapy was also noted but it was not as pronounced as after six cycles. Lanes 1, 3 and 7 are serum samples from patients diagnosed with grade 1 ovarian cancer before the surgery. Lanes 2, 4 and 8 are the matching serum samples from the same patients after surgery and six cycles of chemotherapy. Lanes 5 and 6 are matching serum samples from a patient diagnosed with grade 1 ovarian cancer before and after surgery and two cycles post chemotherapy. (b) Quantification of irILK expression in the serum samples was performed by densitometry and expressed as peak OD±SEM of the number of samples described above. (c) The quantitative determination of CA 125 levels in the matching serum samples of patients with grade 1 ovarian cancer.

These results provide evidence of the potential role of irILK as a biomarker for not only early stage screening but also depicts its utility in clinically monitoring patients after chemotherapy.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A cell-free immunoreactive Integrin Linked Kinase (ILK).

2. An immunoreactive Integrin Linked Kinase according to claim 1, wherein the kinase has a molecular weight of roughly 59 kDA as determined by SDS PAGE.

3. An immunoreactive Integrin Linked Kinase according to claim 1, wherein the kinase is detectable in mammalian serum by Western blotting with a polyclonal antibody raised against the kinase domain of ILK.

4. An immunoreactive Integrin Linked Kinase according to claim 1, wherein the kinase is detectable in mammalian (a) peritoneal fluid by Western blotting with immunoaffinity-purified polyclonal anti-ILK corresponding to the kinase domain of human ILK followed by peroxidase labelled secondary antibody, (b) tissue-conditioned medium by Western blotting using ILK antibody; or (c) serum or peritoneal fluid by immunoprecipitation on protein in 95% acetone:ethanol (1:1) with capture by antibody against ILK and immunoprecipitation visualized by Western blotting.

5. The ILK according to claim 1, which is a biomarker for cancer.

6. A method of detection of cancer, comprising determining the presence or absence of immunoreactive ILK (irILK) in a sample of a biological fluid from a subject, wherein the presence of irILK is an indication of cancer.

7. A method according to claim 6, for the efficacy of a treatment for cancer, comprising carrying out periodic tests, each test comprising determining the concentration or activity of irILK in a sample of a biological fluid from a subject, wherein a decrease in irILK concentration or activity between tests is indicative of the efficacy of any treatment.

8. A method according to claim 6, for detecting recurrence of cancer, comprising determining the presence or absence of irILK in a sample of a biological fluid from a subject who has had cancer, wherein the presence of irILK indicates recurrence of cancer.

9. A method according to claim 6, for assessing the severity of cancer, comprising determining quantitatively the amount or activity of irILK in a sample of a biological fluid from a subject, and correlating results with those previously determined for various grades or stages of cancer or with one or more other markers of cancer.

10. A method according to claim 9, wherein the other marker of cancer is CA125.

11. A kit for carrying out the method of claim 6, said kit comprising means for detecting irILK in biological fluid.

12. A kit according to claim 11, comprising an anti-ILK antibody.

13. A kit according to claim 11 for the diagnosis of cancer or determining the efficacy of treatment of cancer.

14. (canceled)

15. A method of treatment or prevention of cancer comprising administering to a patient determined as suffering therefrom or at risk of developing cancer an agent capable of blocking the increased expression of ILK, or the effect of increased expression of ILK.

16. (canceled)

17. A method according to claim 15, wherein the agent capable of blocking the increased expression of ILK comprises an antisense nucleic acid for use in a gene therapy approach.

18. A method according to claim 15, wherein the agent capable of blocking the effect of increased expression of ILK comprises an anti-ILK antibody or an antagonist of ILK activity.

19. A pharmaceutical composition for use in treating cancer which comprises an agent capable blocking the increased expression of ILK, or the effect of increased expression of ILK, and a pharmaceutically acceptable carrier.

20. The method according to claim 6, wherein the sample of biological fluid comprises whole blood, plasma, serum or peritoneal fluid, ascites, or medium used to perfuse ovarian biopsies, termed tissue conditioned medium.

21. A method according to any one of claim 6, wherein the cancer is present in a mammal.

22. A method according to claim 21, wherein the mammal is a human.

23. A method according to claim 15, wherein the cancer is one in which ILK expression is increased.

24. A method according to claim 23, wherein the cancer is selected from Ewing's sarcoma, primitive neuroectodermal tumor, medulloblastoma, neuroblastoma, prostate cancer and colon cancer.

25. A method according to claim 23, wherein the cancer is ovarian cancer.
